(19) United States
(12) Patent Application Publication
(10) Pub. No.: US 2006/0074324 A1
(43) Pub. Date: Apr. 6, 2006

(54) Biosensing meter plus blood pressure measuring apparatus

(76) Inventors: Shu-Mei Wu, Taipei City (TW); Chia-Chi Wu, Kaohsiung City (TW); Tung-Chuang Jan, Taipei City (TW); Chao-Wang Chen, Taipei City (TW)

Correspondence Address: HDSL, 4331 STEVENS BATTLE LANE, FAIRFAX, VA, 22033, US

(21) Appl. No.: 10/958,287

(22) Filed: Oct. 6, 2004

(51) Int. Cl. A61B 5/02; C12Q 1/54; A61B 5/00

(52) U.S. Cl. 600/490; 600/549; 600/365; 435/14

(57) ABSTRACT

A biosensing meter plus blood pressure measuring apparatus has a slot, an inflatable and deflatable cuff, a pressure sensor and a microprocessor. The slot is able to accept a code card with multiple parameters or a sample strip that comprises a reaction well and a plurality of electrodes thereon in contact with the reaction well. The pressure sensor is used for detecting arterial counterpressure pulses and oscillations at each of the cuff pressure deflating process. The microprocessor is used for controlling the cuff, converting counterpressure pulses and oscillations to voltage signals, processing the voltage signals into a sequence of peak amplitudes and determining a systolic pressure and a diastolic pressure. Furthermore, the microprocessor is used for reading multiple parameters in the code card to measure an analyte in an analyte-containing fluid and calculating a concentration of the analyte. When an analyte-containing fluid (normally blood) is received on the sample strip, the inspection result is obtained according to the operation procedure and parameters obtained previously by the code card. The present apparatus can monitor the blood glucose and blood pressure values at the same apparatus.

### BACKGROUND OF THE INVENTION

1. Field of the Invention

The present invention relates in general to a biosensing meter plus blood pressure measuring apparatus. More particularly, the present invention relates to an electrochemical glucose biosensor plus blood pressure measuring apparatus.

2. Description of the Related Art

In recent years, many studies indicate that diabetes mellitus and hypertension have high relationship with each other. Therefore, patients with diabetes mellitus or hypertension maybe need to monitor blood glucose and blood pressure at the same time. Since modern people pay attention to health and the improvement of the medical treatment, the concept of nursing in house is important. Even patients do not stay in a hospital, patients can monitor their physiological value in house to monitor chronic diseases. A conventional biosensing meter and a blood pressure measuring apparatus are used to measure blood glucose and blood pressure without professional persons operating these apparatus. Furthermore, these apparatuses are designed to simply operate by user with an operation manual.

In the past, various techniques and devices have been used for measuring one or more of blood pressure values. The most common method involves applying a pressure cuff about the upper arm of the human and inflating it so as to stop the flow of blood in the brachial artery. The pressure is then slowly relieved while a stethoscope is used on the distal portion of the artery to listen for pulsating sounds, known as Korotkoff sounds, which accompany the reestablishment of blood flow in the artery. As the pressure in the cuff is reduced further, the Korotkoff sounds eventually disappear. The cuff pressure at which the Korotkoff sounds first appear during deflation of the cuff is a measure of the systolic pressure and the pressure at which these sounds disappear is a measure of the diastolic pressure. This method of blood pressure detection is generally known as the “auscultatory method”. Furthermore, various devices are well known in the prior art for automatically performing blood pressure measurement by the auscultatory method. These devices employ a pump to automatically inflate a pressure cuff and a microphone to convert the Korotkoff sounds into electrical signals which are easily detected by various types of circuits.

In addition, the conventional biosensing meter applied for detecting the substance contained in the blood to be analyzed, such as glucose or cholesterol normally employs a disposable sample strip is to complete the inspection. The sample strip has a reaction well to allow blood dripped thereon. Via the combination of microprocessor/ROM, the whole operation is controlled. Further by execution various procedures, the analysis results for measurement are obtained.

However, since the conventional biosensing meter and the conventional blood pressure measuring apparatus are belong to different apparatuses, users will use two different apparatus to measure blood glucose and blood pressure. If users will go out to play, the users need to bring two apparatuses that will achieve measurement aim. It is very inconvenient to bring two apparatuses. Furthermore, two measurement data of blood glucose and blood pressure recorded in two different apparatus will waste storage space and the data may be lost easily. In addition, the two measurement data do not compare easily with each other for medical monitoring.

### SUMMARY OF THE INVENTION

The present invention provides a biosensing meter plus blood pressure measuring apparatus which can measure a substance contained in blood, e.g. blood glucose and cholesterol, and the blood pressure at the same apparatus. The present apparatus has multiple advantages that are having two measuring functions, having small volume, bringing easily and having inexpensive cost.

Accordingly, the biosensing meter plus blood pressure measuring apparatus in accordance with the present invention comprises a slot, a cuff, a pressure sensor and a microprocessor. The slot is able to accept a code card with multiple parameters or a sample strip that comprises a reaction well and a plurality of electrodes thereon in contact with the reaction well. The cuff may be inflatable and deflatable. The pressure sensor is coupled with the cuff for detecting arterial counterpressure pulses and oscillations at each of the cuff pressure deflating process. The microprocessor is used for controlling the cuff inflating and deflating, converting counterpressure pulses and oscillations to voltage signals, processing the voltage signals into a sequence of peak amplitudes and determining a systolic pressure and a diastolic pressure. Furthermore, the microprocessor is also used for reading multiple parameters in the code card for controlling the operational procedure to measure an analyte in an analyte-containing fluid and determining a concentration of the analyte.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a perspective view of a biosensing meter plus blood pressure measuring apparatus in accordance with the present invention;

FIG. 2 is a partial exploded perspective view of the biosensing meter plus blood pressure measuring apparatus of FIG. 1;

FIG. 3 is a block diagram of the biosensing meter plus blood pressure measuring apparatus of FIG. 1;

FIG. 4 shows the biosensing meter plus blood pressure measuring apparatus of FIG. 1 applied to measure blood glucose; and

FIG. 5 shows the biosensing meter plus blood pressure measuring apparatus of FIG. 1 applied to measure blood pressure.

### DESCRIPTION OF THE PREFERRED EMBODIMENTS

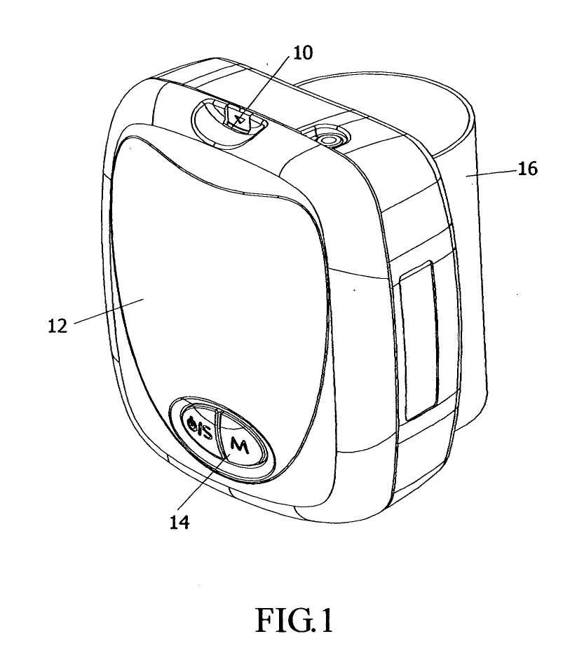

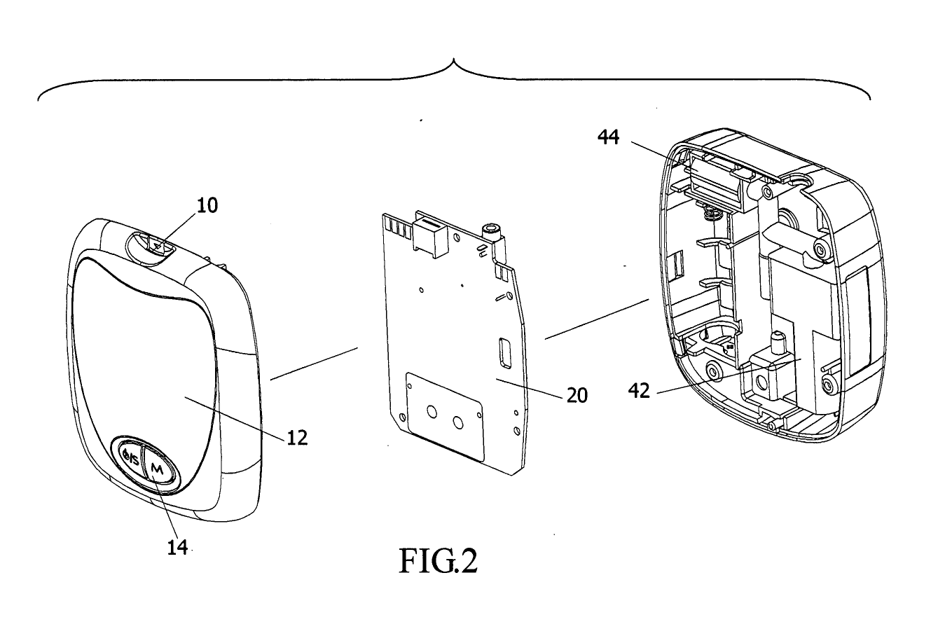

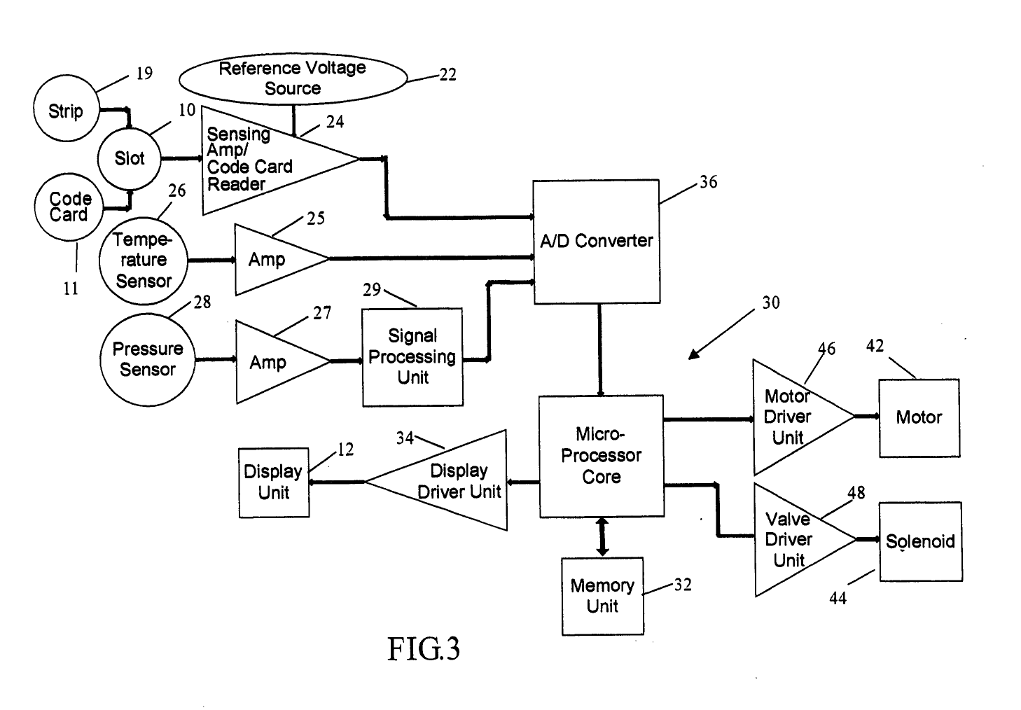

With reference to FIGS. 1 to 3, a preferred embodiment of a biosensing meter plus blood pressure measuring apparatus in accordance with the present invention comprises a slot (10), an operational display unit (12), at least one operational key (14), a cuff (16), a circuit board (20), an operational motor (42) and an operational solenoid (44).

The slot (10) accepts at least one sample strip (19). The sample strip (19) comprises a reaction well that contained an analyte, and multiple electrodes contacted with the reaction well. Preferably, the slot (10) may also accept a code card (11). The code card (11) comprises multiple parameters for upgrading the biosensing meter plus blood measuring apparatus. Furthermore, the code card (11) may further comprise a resister for calibration of the apparatus. In additional, a check card may insert into the slot (10) for checking the state of the apparatus. Preferably, the slot (10) identifies different card or strip by circuit design. In another embodiment of the present invention, the apparatus may further comprise a second slot that can accept the code card (11).

The display unit (12) may show measurement results, some parameters or operation procedures for controlling operations to make users easily manipulate. The key (14) may be used to choose operation modes, operate measurement or open power. The cuff (16) is inflated or deflated for measuring blood pressure. When users will measure blood pressure, users can cover the cuff (16) on the upper arm or the wrist of the users to measure blood pressure.

The circuit board (20) controls the measurement of analyte, e.g. blood glucose, or blood pressure. In an embodiment of the present invention, the circuit board (20) comprises a reference voltage source (22), a temperature sensor (26), a pressure sensor (28), a microprocessor (30) and a memory unit (32).

The reference voltage source (22) provides an operation voltage to the sample strip (19) and the sample strip (19) produce a result transmitting to the microprocessor (30) when an analyte-containing fluid has been dripped in the reaction well of the sample strip (19). Preferably, a sensing amplifier/code card reader (24) is coupled to the slot (10). The sensing amplifier of the sensing amplifier/code card reader (24) connects to the electrode of the sample strip (19) when the sample strip (19) is inserted into the slot (10). When an analyte-containing fluid, for example, a drop of blood, has been dripped in the reaction well of the sample strip (19), an output signal corresponding to a sensing current is generated. If the code card (11) is inserted into the slot (10), the code card reader of the sensing amplifier/code card reader (24) reads the multiple parameters of the code card (11) transmitting to the microprocessor (30).

The temperature sensor (26) detects a temperature when the measurement is proceeding and obtains a parameter for compensating measured data between different conditions dependent on the temperature. The temperature sensor (26) may connect to an amplifier (25) to amplify an output signal from the temperature sensor (26) and transmit to the microprocessor (30). Otherwise, if the signal of the temperature sensor (26) is enough to detection, the temperature sensor (26) can directly connect to the microprocessor (30). In addition, the pressure sensor (28) detects arterial counterpressure pulses and oscillations at each of the cuff pressure deflating process. Furthermore, the pressure sensor (28) may connect to an amplifier (27) for amplifying signals from the pressure sensor (28). The amplifier (27) connects to a signal processing unit (29) which converts the counterpressure pulses and oscillations to voltage signals. In an embodiment of the present invention, the signal processing unit (29) may be manufactured in the microprocessor (30). Preferably, the pressure sensor (28) may connect directly to the signal processing unit (29) for converting the counterpressure pulses and oscillations to voltage signals.

The memory unit (32) may store multiple parameters and operational procedures from the code card (11). In an embodiment of the present invention, the memory unit (32) preferably is electrically erasable programmable read-only memory (EEPROM).

In an aspect of the present invention, the microprocessor (30) controls the cuff (16) inflatable and deflatable. Preferably, the microprocessor (30) further comprises a motor driver unit (46) and a valve driver unit (48). The motor driver unit (46) connects to the motor (42) and the valve driver unit (48) connects to the solenoid (44). Therefore, the microprocessor (30) controls the motor (42) and the solenoid (44) to inflate and deflate the cuff (16) for measuring blood pressure.

Furthermore, the microprocessor (30) is also used for processing the voltage signals into a sequence of peak amplitudes at each pressure deflating process, and the microprocessor (30) determines a systolic pressure and a diastolic pressure. The microprocessor (30) reads the parameters of the code card (11) and controls the operation procedure for determining an analysis result of the analyte. In an embodiment of the present invention, the microprocessor (30) further comprises a second memory unit, a display driver unit (34) and an A/D converter unit (36). The second memory unit is used for storing the analysis result.

In an embodiment of the present invention, the sensing amplifier/code card reader (24), the amplifier (25, 27), the A/D converter (34), the motor driver unit (46) and the valve driver unit (48) may be manufactured in one microprocessor (30).

In another embodiment of the present invention, the apparatus in accordance with the present invention further comprises a second microprocessor. The microprocessor (30) and the second microprocessor may respectively control the measurement of the analyte and blood pressure. Furthermore, the apparatus according to the present invention further comprises a voice communication unit for communication.

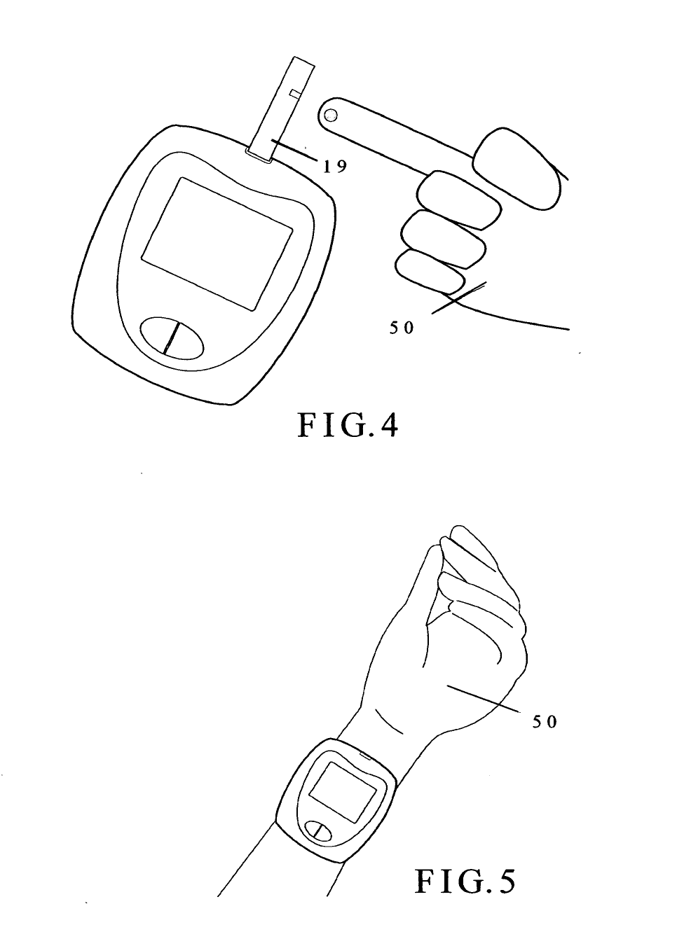

With further reference to FIG. 4, the apparatus according to the present invention is used to measure an analyte, e.g. blood glucose. The code card (11) is inserted into the slot (10) and the parameters in the code card (11) are read by the microprocessor (30). Furthermore, the code card (11) may comprise a resister for calibration of the apparatus. Another, a check card may insert into the slot (10) for checking the apparatus is normal to proceed the measurement.

The reference voltage source unit (22) provides an operation voltage to the sample strip (19) electrode, and the sensing amplifier (24) is connected to another inspection sample strip electrode. When an analyte-containing fluid, for example, a drop of blood from user's finger of his hand (50), has been received in the reaction well of the sample strip (19), an output signal corresponding to a sensing current is generated. With responding to the operation procedure and parameters obtained from the code card (11), the microprocessor (30) determines a plurality of voltages with a predetermined lasting time. The voltages and the lasting time are obtained from the data stored in the code card (11). The microprocessor (30) controls the sensing amplifier (24) to provide a plurality of signal outputs after a predetermined time, so as to display an analysis result measured from the analyte-containing fluid.

With further reference to FIG. 5, the apparatus in accordance with the present invention is used to measure blood pressure. The cuff (16) is covered about the wrist of user's hand (50) and inflating it so as to stop the flow of blood in the brachial artery. The microprocessor (30) controls cuff pressure so as to inflate the cuff (16) to a maximum cuff pressure above a mean arterial pressure of a detected subject. The pressure sensor unit (28) monitors arterial counterpressure pulses and oscillations at each of the cuff pressure deflating process and the signal processing unit (29) coupled to the pressure sensor unit (28) for converting the counterpressure oscillations to voltage signals. The microprocessor (30) processes the voltage signals into a sequence of peak amplitudes at each pressure deflating process, the sequence being enveloped by a curve containing a maximum value and determines a diastolic pressure and a mean arterial pressure based upon the curve and a systolic pressure based upon the diastolic pressure, the mean arterial pressure and the counterpressure pulses.

The biosensing meter plus blood pressure measuring apparatus in accordance with the present invention has following advantages.

(a) The present apparatus may comprise one microprocessor, display unit, EEPROM to measure both blood glucose and blood pressure for decreasing cost and volume.

(b) Since the present apparatus can measure blood glucose and blood pressure and diabetes mellitus and hypertension have high relationship with each other, the measurement result can provide for clinical observation and personal medical control.

(c) The present apparatus can record results and time and also can wireless or wired connect to personal computer for analysis.

(d) The present apparatus may store the blood glucose and blood pressure result in one memory unit so the utilization of the memory unit is increasing.

(e) The present apparatus may increase a voice communication function so the function of the apparatus is increasing.

(f) The sample strip and the code card can insert into the same slot so the cost is decreasing and the manufacturing method of the present apparatus is simple.

Other embodiments of the invention will appear to those skilled in the art from consideration of the specification and practice of the invention disclosed herein. It is intended that the specification and examples to be considered as exemplary only, with a true scope and spirit of the invention being indicated by the following claims.

What is claimed is:

1. A biosensing meter plus blood pressure measuring apparatus comprising:
a code card stored a plurality of parameters therein;
a sample strip comprising a reaction well and a plurality of electrodes thereon in contact with the reaction well, and
a slot for receiving the code card or the sample strip;
an inflatable and deflatable cuff;
a pressure sensor coupled with the cuff for detecting arterial counterpressure pulses and oscillations at each cuff pressure deflating process during a blood pressure measurement; and
a microprocessor for controlling the cuff inflating and deflating, converting the counterpressure pulses and oscillations to voltage signals, processing the voltage signals into a sequence of peak amplitudes and determining a systolic pressure and a diastolic pressure; and reading the parameters in the code card for controlling operational procedures to measure an analyte in an analyte-containing fluid and determining a concentration of the analyte.

2. The biosensing meter plus blood pressure measuring apparatus as claimed in claim 1 further comprising a second slot for receiving the code card.

3. The biosensing meter plus blood pressure measuring apparatus as claimed in claim 1 further comprising a second microprocessor, wherein the microprocessor and the second microprocessor respectively control the measurement of the analyte and the blood pressure.

4. The biosensing meter plus blood pressure measuring apparatus as claimed in claim 1 further comprising a display unit.

5. The biosensing meter plus blood pressure measuring apparatus as claimed in claim 1, wherein the analyte is blood glucose.

6. The biosensing meter plus blood pressure measuring apparatus as claimed in claim 1, wherein the microprocessor further comprises a motor driver unit for controlling the cuff to inflate and a valve driver unit for controlling the cuff to deflate.

7. The biosensing meter plus blood pressure measuring apparatus as claimed in claim 6 further comprising a motor connected with the motor driver unit for inflating the cuff and a solenoid connected with the valve driver unit for deflating the cuff.

8. The biosensing meter plus blood pressure measuring apparatus as claimed in claim 1 further comprising a temperature sensor.

9. The biosensing meter plus blood pressure measuring apparatus as claimed in claim 1 further comprising a check card inserted in the slot, wherein the check card comprises a resister for calibration.

10. The biosensing meter plus blood pressure measuring apparatus as claimed in claim 1, wherein the code card further comprises a resister for calibration.

11. The biosensing meter plus blood pressure measuring apparatus as claimed in claim 1 further comprising a memory unit for storing the parameters and the operational procedures from the code card.

12. The biosensing meter plus blood pressure measuring apparatus as claimed in claim 11, wherein the memory unit is electrically erasable programmable read-only memory.

13. The biosensing meter plus blood pressure measuring apparatus as claimed in claim 1 further comprising a voice communication unit.

14. The biosensing meter plus blood pressure measuring apparatus as claimed in claim 1 further comprising a reference voltage source for providing a reference voltage to the electrodes of the sample strip.

15. The biosensing meter plus blood pressure measuring apparatus as claimed in claim 1, wherein the microprocessor further comprises a sensing amplifier connected to the slot.

16. The biosensing meter plus blood pressure measuring apparatus as claimed in claim 1, wherein the microprocessor further comprises a code card reader connected to the slot.

17. The biosensing meter plus blood pressure measuring apparatus as claimed in claim 1, wherein the microprocessor further comprises a memory unit, a display driver unit and an A/D converter unit.

18. The biosensing meter plus blood pressure measuring apparatus as claimed in claim 1, wherein the microprocessor further comprises a signal processing unit for converting the counterpressure pulses and oscillations to the voltage signals.

19. The biosensing meter plus blood pressure measuring apparatus as claimed in claim 1, wherein the microproessor further comprises a plurality of amplifiers for amplifying respectively signals of a temperature sensor and a pressure sensing unit.

20. The biosensing meter plus blood pressure as in claim 1 further comprising at least one key.
